(19) United States
(12) Patent Application Publication
(10) Pub. No.: US 2005/0203410 A1
(43) Pub. Date: Sep. 15, 2005

(54) Methods and systems for ultrasound imaging of the heart from the pericardium

(75) Inventor: David A. Jenkins, Flanders, NJ (US)

Correspondence Address: HELLER EHRMAN WHITE & MCAULIFFE LLP, 1717 RHODE ISLAND AVE, NW, WASHINGTON, DC, 20036-3001, US

(73) Assignee: EP MedSystems, Inc., West Berlin, NJ (US)

(21) Appl. No.: 10/997,874

(22) Filed: Nov. 29, 2004

Related U.S. Application Data
(60) Provisional application No. 60/548,102, filed on Feb. 27, 2004.

(51) Int. Cl. A61B 8/00; A61B 8/12; A61B 8/14

(52) U.S. Cl. 600/459

(57) ABSTRACT

A peritoneal ultrasound imager includes an elongated body less than about 20 inches in length that is adapted to be inserted through a cannula into or near the pericardium space, and an ultrasound transducer array at one end of the body that is suitable for ultrasound echocardiography. The cannula and ultrasound imager may be of a single piece construction. A method for imaging the heart includes introducing a cannula into the wall of a patient's chest, inserting the elongated body into the cannula, moving the inserted elongated body to a position near the heart, and imaging the heart with ultrasound echo.

### RELATED APPLICATIONS

The present application claims benefit of and priority to U.S. Provisional Application No. 60/548,102 entitled METHODS AND SYSTEMS FOR ULTRASOUND IMAGING OF THE HEART FROM THE PERICARDIUM, filed Feb. 27, 2004, which is hereby incorporated by reference in its entirety.

### BACKGROUND OF THE INVENTION

1. Field of the Invention

The present invention is directed at systems for examining a heart, and more particularly to a method and apparatus for imaging the heart using an ultrasound imaging catheter.

2. Description of the Related Art

Cardiac monitoring and cardiac intervention are important procedures in modern medicine. Information intensive procedures such as cardiac imaging generally requires placing one or more sensors at or in the heart itself, requiring some degree of invasiveness. High resolution heart imaging, for example, often is done by inserting an ultrasound imaging catheter into the heart via the femoral artery.

One recent technique in this area, known as “cardiac resynchronization therapy” sometimes uses a thoracoscopic approach as a minimally invasive technique for procedures involving electrical lead placement, as summarized by Steinberg et all, PACE 26: 2211-2212 (2003). However, this approach has been said to be limited by lack of site-directed imaging, which is needed for optimal LV lead placement. Steinberg et al., PACE 26: 2212 (2003).

Percutaneous catheter-based methods of monitoring and therapeutic intervention can be very expensive for several reasons. The procedure of opening the body at a location removed from the heart, typically in the right leg, and snaking a catheter through an artery a long distance to the heart requires some time. The long catheters used in such procedures, which generally are disposable, can be exceedingly expensive. Highly advanced catheters such as 2-dimensional ultrasound phased array imaging catheters developed by EP MedSystems, Inc. feature long coaxial cables, that are a great improvement due to their greater immunity to spurious signals, DC voltages and cross talk, but that may be expensive. Accordingly, less expensive and more convenient tools are desired in this field.

Cardiac imaging performed by ultrasound imaging catheters generally involve positioning the imaging portion of the catheter within the right atrium. So positioned, structures of the left atrium and left ventricle are near or beyond the maximum resolving distance for ultrasound imaging, which is limited by the attenuation of ultrasound energy in blood and heart tissue. Yet several clinical procedures require accurate imaging of the left ventricle wall, such as for example cardiac resynchronization therapy (CRT), which requires identifying the last contracting myocardial segment. Modification of the left atrial appendage in a minimally invasive manner also requires better imaging than can be provided by a catheter in the right atrium. Thus, current intracardial ultrasound imaging methods may not provide the optimum level of image resolution to support important therapies.

The catheter currently manufactured by EP MedSystems is a 9-French size, which, in an adult patient can easily be placed in the right atrium for intracardiac imaging. However, in neonates and pediatric patients, this size may be too large to manipulate through the vascular system into the heart. Thus, another approach to close range, and hence, higher resolution imaging of the heart in this group of patients is needed.

Additionally, it should be noted that imaging is potentially only one piece to an overall minimally invasive heart procedure. Ablation of the tissue at or near the ostia of the pulmonary veins in the left atrium requires a number of catheters to be placed in the heart: one in the coronary sinus, one to measure conduction in high right atrium, one to pace or defibrillate in the right ventricle, to name the more common catheters. In newer versions of this procedure, even a “basket” or “balloon” catheter with, for example, 64 electrodes to map the conduction of the heart in a single beat, is utilized. All of these other necessary tools take up space within both the accessible vasculature and chambers of the heart. Thus, while it also may be necessary to place an ultrasound-imaging catheter into the heart, this may be seen as a luxury which cannot be effectively utilized. Less effective imaging, such as transesophageal ultrasound, may be used instead. Examples of other procedures which also use minimally invasive catheter tools, and thereby utilizing the small amount of available space, include heart valve repair or replacement, atrial septal repair, left atrial appendage modification, and removal of pacemaker leads. Thus, there is a need to complement the various minimally invasive heart procedures now coming of age.

Thus, there is a need for methods and devices for imaging the heart less expensively and imaging the structures of the heart, especially the left ventricle and left atrium, with greater sensitivity and resolution than is achievable using conventional techniques and devices.

### SUMMARY OF THE INVENTION

Embodiments reduce the cost of an ultrasound imaging catheter by providing a much shorter catheter that is introduced into the body much closer to the heart.

A new therapy to treat heart failure is bi-ventricular pacing, or “resynchronization” therapy, where both ventricles of the heart are paced with an implantable pulse generator, commonly known as an artificial pacemaker. Normal pacing for a slow heart is performed via an implanted electrode in the right ventricle. The conduction myofibers (Purkinje fibers) conduct the electrical pulse and the ventricles contract synchronously in an inward direction, resulting in blood being pumped efficiently from the heart. In heart failure, the left ventricle becomes enlarged and conduction through the tissue of the left ventricular wall often becomes slow, so that the upper part of the left ventricle conducts as much as 200 to 250 milliseconds behind the apex area of the ventricles. This leads to poor and discoordinated contraction, and in many cases, an outward movement of the heart muscle, so that blood sloshes around rather than being squeezed out of the ventricle. Thus, an ideal location to place a pacing electrode in the left ventricle is in the area of slowest conduction, which can be a rather large area of the left ventricle, and may not always be the area that has the largest conduction. The problem facing physicians today is to locate the optimal spot for the permanent fixation of the pacing electrode. The thrust of this invention is to provide a method and device to optimize the location of the electrode.

A normal pacemaker electrode is ideally implanted in a location which achieves the lowest “threshold,” which is the lowest voltage level to excite the surrounding tissue to synchronously conduct the pacing signal from the electrode. Thus, the electrode is implanted based upon merely finding the spot with the lowest voltage that “captures” the tissue. With heart failure, in the left ventricle, it is not so simple. Capture may not be the best parameter to use. Furthermore, advancing the electrode to the proper spot may not be easy. What is most desired is to optimize EF, while the threshold for “capture” is really secondary. Thus the ability to not only visualize the motion of the left ventricular wall, but also measure EF, or some form of output of the heart, such as stroke volume, flow rate, or ventricular wall motion is highly desirable during the implantation procedure. This invention puts forth the use of ultrasound technology for this purpose.

Ultrasound is well known as an imaging tool. However, imaging through the chest is very difficult in that the ribs block the view and that the depth of penetration gives poor resolution. Ideally, the ultrasound transducer should be positioned closer to the heart. An esophageal ultrasound probe has been used on more than 50 patients in an attempt to view the heart. See, e.g., Jan et. al., Cardiovasc. Intervent. Radiol., 24, 84-89 (2001). Unfortunately, the results are less than desired since the probe must view through the esophagus and both walls of the heart, lending to less resolution in the image than desired. Intravascular ultrasound systems, although ideal in its size with thin catheters, generally utilize with high frequencies which result in poor depth of penetration. X-ray or X-ray fluoroscopy may give good images of the electrode, but not of the actual tissue of the heart (most particularly the walls of the ventricle).

The present invention overcomes these problems. Preferably, the present invention uses an ultrasound imaging catheter for viewing from the outside of the heart, via an incision through the chest of a patient. This catheter would connect either directly to a display system or through a connecting cable, as shown in FIG. 6. The ultrasound display can provide a display of the measurement of cardiac output in assisting the physician with the procedure.

In an embodiment, a peritoneal ultrasound imager includes an elongated body having a length less than about 20 inches that is adapted to be inserted through a cannula into the peritoneal space, and an ultrasound transducer array coupled at the distal end of the elongated body that is suitable for ultrasound echocardiography. The cannula and ultrasound imager may be of a single piece construction. The ultrasound transducer may be made up of multiple piezoelectric transducers (such as one of 48, 64, 96, or 128 transducer elements) configured as a linear phased array, each connected to a coaxial cable that can be connected to a coupling circuit that may provide electrical isolation. The elongated body may be rigid and can be manipulated within a patient's body by moving a portion extending outside the cannula. The elongated body may also have a portion that is bendable with the bend being controllable from a handle connected the portion extending outside the cannula. The elongated body may also include one or more electrodes. The elongated body may also be configured to be manipulated by a robotic system.

In an embodiment, integrated cannula and imaging catheter include a sheath and an elongated body within the sheath slideably adapted for insertion through a chest wall into a peritoneal space, and an ultrasonic imaging array positioned on the elongated body proximal to the distal tip that is configured for obtaining a two dimensional image. The sheath may include extracorporeal fixation device, located external to the patient to prevent inward movement of the sheath and an internal valve or seal. The integrated cannula and imaging catheter may be configured as a single use, disposable device.

A method for imaging the heart includes introducing a cannula into the wall of a patient's chest or thorax, inserting into the cannula an elongated body having an ultrasound imaging sensor at one end, moving the inserted elongated body to a position near the heart, such as within the pericardium, and imaging the heart with ultrasound echocardiography by emanating ultrasound from the ultrasound imaging sensor and receiving ultrasound echoes with the sensor. The method may be performed in part by a robotic system for manipulating the elongated body.

### BRIEF DESCRIPTION OF THE DRAWINGS

FIG. 1 shows a percutaneous catheter according to an embodiment.

FIG. 2 shows placement of a percutaneous catheter according to an embodiment.

FIG. 3a shows placement of a filled zone according to an embodiment.

FIG. 3b shows detail of a filled zone according to an embodiment.

FIG. 4 shows a percutaneous catheter with a rotatable transducer.

FIG. 5 shows detail of a transducer in a percutaneous catheter.

FIG. 6 shows a percutaneous catheter connected to other equipment according to an embodiment.

### DETAILED DESCRIPTION OF THE EXEMPLARY EMBODIMENTS

The various embodiments of the present invention include a much shortened percutaneous catheter system that is configured to be inserted into the chest cavity through a small opening and thence manipulated to the pericardium. An array of ultrasonic transducers and/or ECG sensors on the catheter desirably are positioned on the outside surface of or inside the pericardium in the vicinity of the heart by means of a cannula inserted in the chest. Positioning advantageously is carried out manually or by robotic or semi-robotic manipulators. Once positioned near the heart, the sensors send signals to other externally positioned electronic equipment attached or otherwise in communication with the catheter.

For example, an array of piezoelectric transducers may be energized to pulse ultrasonic energy and, acting as receivers, detect reflected ultrasound energy, converting received ultrasound into electrical signals (“detected signals.”). The detected signals are conducted to connected externally positioned equipment for processing. Such processing may generate images of tissues, color Doppler images showing motion of tissue (“tissue Doppler images”) or blood, or quantified measurements of movement of tissues and/or blood. Such imaging of structures and tissue/flood movement within the heart by analysis of ultrasound echoes is known as “echocardiography.” For example, pulses may be monitored to produce, as a kind of snapshot, a 2-dimensional image of a planar cross section. One or more ECG electrodes may be present, and used to generate an electrocardiogram of the heart. Other sensors, such as a temperature sensor (e.g., thermistor or thermocouple) also may be included for diagnostic, control or safety purposes.

Cardiac ultrasound imaging, or echocardiography (“ultrasound echocardiography”) desirably creates detailed cardiac, intracardiac, and vascular anatomy images. Doppler echocardiography, for example, relies on the physics of ultrasound transmission to determine the velocity and direction of blood flow, and is used to determine pressure and flow and to visualize blood movement within the cardiac chambers. Diagnostic ultrasound imaging applies high frequency pulsed and/or continuous sound waves to the body and uses computer-assisted processing of the reflected sound waves to develop images of internal organs and the vascular system. The waves may be generated and recorded by transducers or probes that may be inserted into the body. The resulting images can be viewed immediately on a video display or recorded for later evaluation by a physician in continuous or single image formats.

In an embodiment, the catheter is inserted into the chest cavity after first establishing an opening to the cavity via a cannula or chest tube. In another embodiment the cannula or chest tube is integrated with the catheter into a single device, or two part device. Optionally, a deflector, such as a fixed or movable shield made of a plastic or metal may cover the distal opening of the cannula, or chest tube and/or catheter to form a barrier during penetration of tissue. A trocar may be used to establish the opening, with the trocar being a separate tool or integrated with the cannula or chest tube. Desirably, a flange, solid body or other material is attached to a proximal location of the catheter to prevent inward movement past a position. For example, a sheath enveloping the elongated body can be prevented from excessive insertion by a flanged extracorporeal fixation device, having a rim or collar of an average width of at least 2 millimeters, 5 millimeters, 10 millimeters, 15 millimeters, 20 millimeters or more extending around the sheath.

In an embodiment, the imaging catheter is positioned over either the left or right ventricles, or both, in order to image the entire heart by moving the catheter within the pericardium. The imaging catheter may be positioned manually. Robotic positioning, or positioning assistance also may be used. A commercial system may be used or modified for this purpose. For example, the da Vinci Robotic Surgical System (Intuit Surgical Inc., Sunnyvale, Calif.) allows positioning via a surgeon control panel. A robotic system may provide computer interfacing to allow scaled motion, thus alleviating tremor and providing accurate surgical precision through small ports. Robotic assisted manipulation also may be used. In an embodiment, a computer interface allows greater precision and steadiness of positioning, while providing at least partial user muscle derived motion. An example of robotic assisted technology in this context is the work reported by DaRoss et al., J. Am Coll. Cardiol. 41: 1414-1419 (2003). Also, see Steinberg and DeRose PACE 26: 2211-2212 ((2003) entitled “The Rationale for Nontransvenous Leads and Cardiac Resynchronization Devices,” which describes cardiac resynchronization therapy using specialized leads and devices.

Ultrasound Imaging Catheter

A catheter according to embodiments is an elongated member (e.g., tube or rod) with an imaging ultrasound sensor (e.g., a linear phased array transducer) positioned at a distal end and a handle positioned at a proximal end. The catheter may be flexible, inflexible or flexible in part. The total length desirably is less than 50 cm, 35 cm, 30 cm, 25 cm, 20 cm or even less than 15 cm. The width desirably is less than 8 mm, 7, mm, 6 mm, 5 mm, 4 mm, 3 mm, 2.5 mm or even less than 2 mm. The catheter may comprise a wide range of materials, including, for example, nylon, Teflon, polyethylene, other polymer, stainless steel, platinum and other metals.

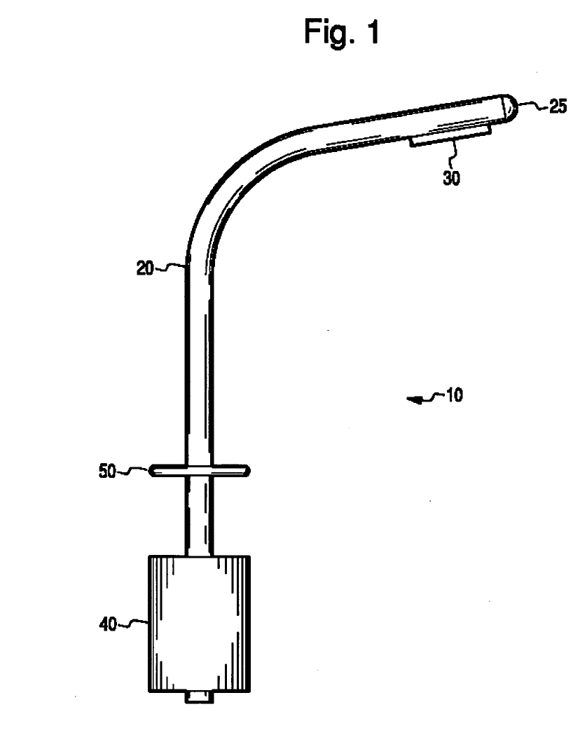

FIG. 1 depicts a representative catheter shape. In this figure, catheter 10 with distal section 20 and distal tip 25 has a linear phased array ultrasonic transducer at spot 30, and can be manipulated by handle 40, which remains outside of the body by virtue of extracorporeal fixation device 50. During use, distal tip 20 is pushed through a cannula or chest tube having a seal (not shown), so that a portion of the catheter body, extending up to the extracorporeal fixation device 50, enters the interperitoneal space of the thorax of a patient (not shown). Also not shown are optional ECG electrodes, which may be present as, for example gold patches or rings around the catheter. A metallic surface also may be present and serve as a sensor or stimulatory electrode.

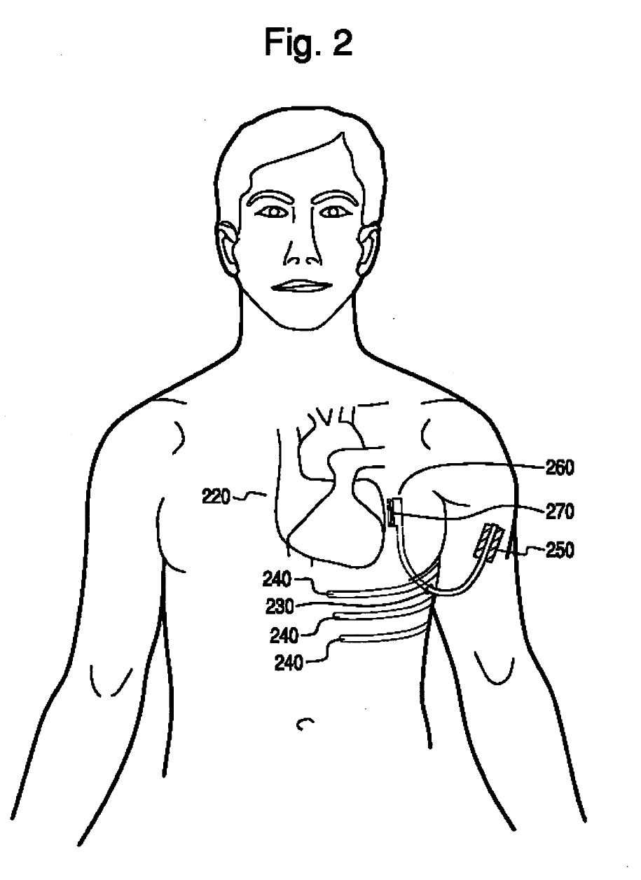

FIG. 2 depicts a representative positioning of the catheter 210 of FIG. 1 after inserted into an interior body space surrounding the heart 220 through cannula or chest tube 230, that typically may be located between adjacent ribs 240. Handle 250 is manipulated to move the distal tip 260 around to the left side of the patient's heart 220 (seen as the right side in this figure). Once there, sensor array 270 and/or stimulators (not shown) on the catheter may be activated.

A variety of sensors useful in embodiments readily will be appreciated by a skilled artisan. Most desirably, multiple ultrasonic transducers, such as an array of transducers capable of being used as a phased array may be employed. A transducer alternately may comprise an annular array of transducer elements. In one aspect, the annular array defines a face that is generally elliptical in shape. In another, the annular array defines a face that is generally circular in shape. The face may be generally flat or have a spherical or other curvature.

In an embodiment, a linear phase array of piezoelectric transducers is positioned along the long axis of the catheter near the distal end. Advantageously, multiple piezoelectric devices emit sonic vibrations sequentially along the axis by selective interference and reinforcement of sound waves to generate narrow sound beams. Such phase-reinforced beams can be shifted by adjusting the phase lag between elements so as to store the beam through a large angle (scan angle). Echo information collected by the piezoelectric elements for each beam position can then be correlated to create a 2-dimensional field of echo information within the scan angle. This information can be used to create a 2-dimensional image parallel to the long axis of the array, within the scan angle to the maximum distance from which echo information can be received.

In an embodiment, the percutaneous catheter comprises a “hooked shape,” wherein at least the portion with an attached ultrasonic imaging array has a different axis than a proximal handle region that is graspable by a user or robotic device for manipulation. The imaging array region or the graspable region or both may be curved, and one or both regions may be on linear segments that do not share the same vector in space. In each case, the percutaneous catheter is said to have a “hooked shape.” In an embodiment, the hook shape is characterized by a change in vector, proceeding from the distal tip to the spot that penetrates the body wall, of between 5 degrees to 170 degrees, advantageous between 10 degrees to 120 degrees and more desirably between 20 degrees and 110 degrees. Typically the attached ultrasound imaging array is in a linear form having an axis that differs between 5 degrees and 120 degrees from the axis of a handle region and more advantageously is between 15 degrees and 75 degrees different.

A connecting region of the elongated body between the attached ultrasonic array region and a handle region may comprise one or more discontinuous bends, or may be curved. The imaging array is located at or near the distal end of the catheter. In an embodiment, the nearest edge of the imaging array is between 0.1 mm and 50 mm from the distal tip of the catheter, more desirably between 0.5 mm and 25 mm away, and yet more desirably between 1 mm and 15 mm away.

The catheter may be steered in two dimensions in an imaging plane. In an embodiment a catheter linear phase array is positioned on or within a bendable portion of the catheter, such as a portion capable of being bent through an arch having a radius of curvature between 0.25 to 2.5 inches, and more desirably about 1 inch of radius. The curve may be tensioned, for example by a separate tension knob on the handle or by friction. Suitable structures, methods and materials for assembling the bendable portion of the catheter are disclosed in pending U.S. patent application Ser. No. 10/819,358, entitled Steerable Ultrasound Catheter assigned to EP MedSystems, Inc., filed Apr. 7, 2004, which is hereby incorporated by reference in its entirety.

In an embodiment, received echo signals are transferred from the scanner array down the catheter length by coaxial wires. to a high frequency coupler such as a transformer at the proximal end of the catheter. The coupler may transfer information further into a circuit that is interfaced with a computer. A variety of high frequency couplers are contemplated that may be electrically attached to the coaxial cables and configured to electrically isolate direct current between the piezoelectric devices in the body and equipment connected to the catheter outside of the body. Suitable couplers for an isolation circuit are disclosed in U.S. patent application Ser. No. 10/345,806, entitled Ultrasound Imaging Catheter Isolation System With Temperature Sensor, Attorney Docket No. 4426-47, filed Jan. 16, 2003 and assigned to EP MedSystems, Inc., which is incorporated by reference in its entirety.

According to an embodiment, the imager is inserted into a chest cavity and manipulated by grasping a proximal portion outside of the body and moving the elongated body so as to position the imaging array on or near the (e.g. within 2 cm, 1 cm, 0.5 cm, 0.2 cm, 0.1 cm or less) heart. Desirably, the device is positioned outside the outer surface of the pericardium, which covers the heart.

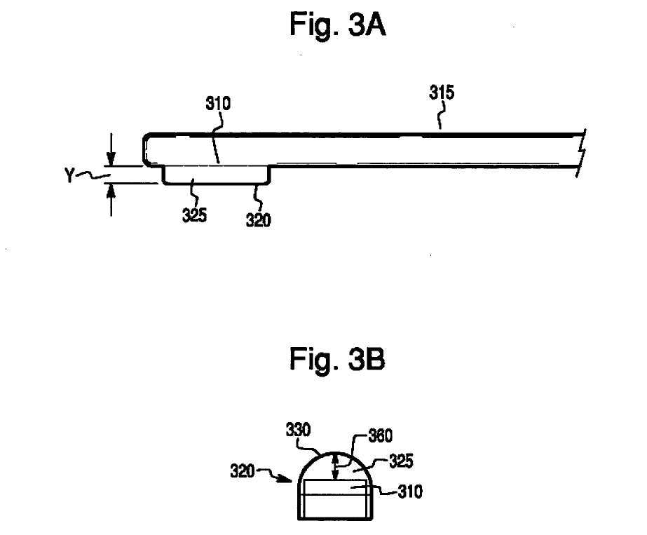

In an embodiment shown in FIGS. 3a and 3b, the ultrasound transducer array surface 310 within elongated body 315 is held a short distance away from a structure to be imaged, such as the exterior surface of the pericardium (not shown), via a covering 320 of filled space 325 at least throughout most (e.g. 50%, 75%, 85%, 95% or more) of surface 310 of the ultrasound transducer array. Desirably, outer surface 320 of filled space 325 is pressed against a structure such as a heart wall or pericardium. Filled space 325 may extend along the length of the ultrasound transducer array as shown in FIG. 3 and may be filled with fluid or solid 325, which may comprise, for example, sterile water, sterile physiological saline, or solid such as a polymer or hydrogel that conducts ultrasound. In an embodiment, filled space 325 is a hydrogel or other body compatible material and lacks distinct covering 320.

In an embodiment, this filled space occupies a zone that keeps an imaged structure away from a transducer by a distance “Y”. Distance Y includes both the thickness 360 of filled space 325 and the thickness of any barrier 330 between the filled space 325 and the outer surface 320, and may be for example, between 0.01 to 50 mm, 0.05 to 10 mm, 0.2 mm to 2 mm, or 0.1 to 5 mm. Filled space 325 can transfer ultrasound from array 310 through distance 360, to the barrier 330 and acoustically couple the ultrasound to barrier 330 so it passes through it to the outer surface 320 where the ultrasound passes into the body. It is believed that filled space 525 may allow positioning of ultrasound transducer array 510 a minimum distance Y from an imaged structure upon placement onto that structure to alleviate near-zone interference, thereby permitting imaging of the entire thickness of the heart wall.

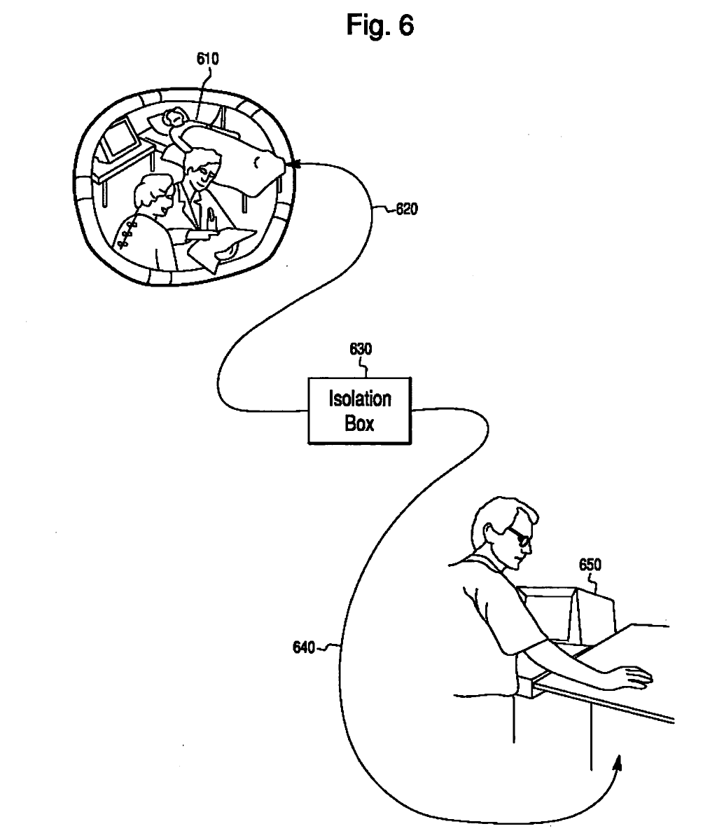

Desirably, as illustrated in FIG. 6, the catheter inserted into the patient 610 is connected by means of a cable 620, 640 to other equipment, such as ultrasound equipment and display monitor 650, via an isolation junction box connector 630 that electrically isolates the patient from the rest of the system. In an embodiment, ultrasound frequencies used are between 2 and 25 MHz, more desirably between 4 and 10 MHz and yet more desirably between 4.5 to 8.5 MHz. The frequencies may be variable by the operator or automatically with variations possible in a stepped manner, for example, at 0.5 MHz intervals.

In an embodiment, the catheter further has an electrically conductive surface of enough area to act as an electrode for administering electroconvulsive shock. In this embodiment, desirably a second electrode is located to be proximate to the other side on the heart. Desirably, the catheter may be placed on the left side of the heart while another electrode, in this embodiment, is positioned on the right side. Such right sided placement could either be within the heart, via a percutaneously placed catheter, or outside the heart, such as a skin patch electrode.

In an embodiment, the ultrasound transducer array may be a linear array of between 4 and 256 transducer elements arranged as a linear phased array. The transducer array may more desirably include between 32 and 128, yet more desirably a 64 element phased array is used for imaging. Ultrasound arrays made up of 48, 64, 96, or 128 transducers are envisioned. The transducer may have an aperture of for example between 3 and 30 mm, and more desirably between 10 and 15 mm. The imaging plane according to an embodiment may be longitudinal side-firing, circularly perpendicular to the catheter axis, or more desirably, longitudinally oriented side firing.

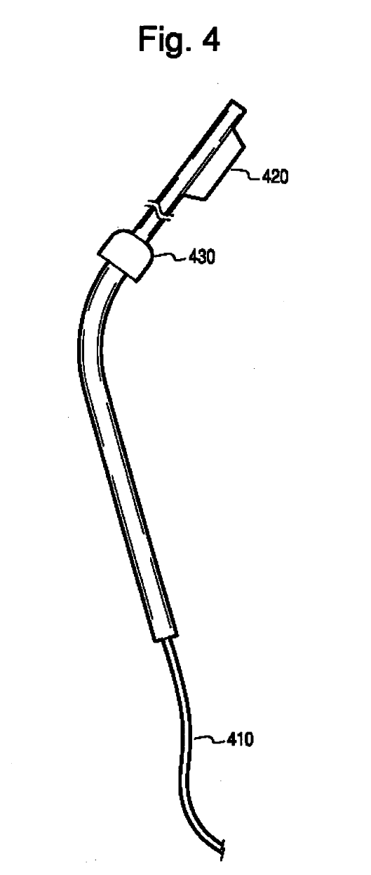

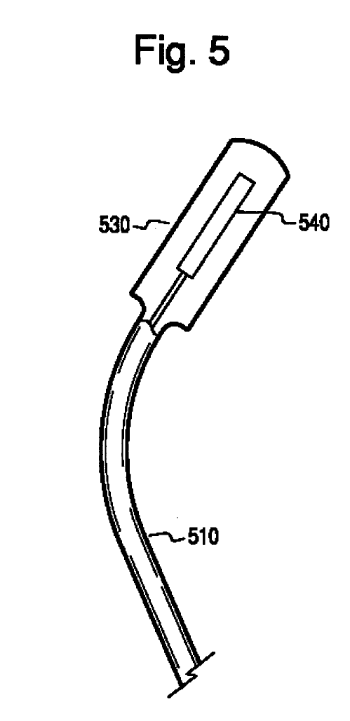

The linear array may be rotated to obtain more space filling information that can be assembled into a meaningful 3-dimensional map and 4-dimensional video images. The imaging catheter may also comprise a drive cable and a gear mechanism configured to position the ultrasound imaging sensor at various angles, with the cable and/or mechanism disposed within a lumen of the catheter body as depicted in FIG. 4. Drive cable 410 as shown in this figure may be coupled to transducer 420 and to gear mechanism 430. The drive cable 410 and gear mechanism 430 are adapted to rotate transducer 420. In this manner, the drive cable and gear mechanism rotate the transducer, about the long axis of the catheter thereby eliminating the need to rotate the catheter body manually to obtain 2-dimensional scans at different angles of rotation. In an embodiment shown in FIG. 5, imaging catheter 510 comprises housing 530 rotatably coupled to its distal end. Transducer 540 is mounted within housing 530 and surrounded by an ultrasound transmitting substance. In such an embodiment, the transducer is rotated relative to the distal end by rotating the housing. Alternatively, the imaging catheter comprises a housing 530 operably attached to a distal end with the transducer 540 being rotatably coupled to the housing. Rotation by at least 5, 10, 15, 30, 40, 45, 55, 65 or more degrees allows capture of multiple 2-dimensional images over several imaging planes, which may then be assembled into 3-dimensional images and/or 4-dimensional moving images.

According to an embodiment of the present invention, a thermistor may be incorporated in or near the transducer 540 that automatically shuts off the catheter assembly at a isolation box. By way of example, an output of the thermistor may be coupled to an enable/disable input to a plurality of gates gating wires passing to/from the transducer elements. So long as the temperature of the catheter assembly remains below a safe level, such as below about 43° C., the gates remain enabled allowing signals to pass to/from the transducer elements. However, should the temperature of catheter assembly reach or exceed an unsafe level, the thermistor disables the gates, automatically shutting off the catheter assembly. Other configurations for automatic shutoff are also contemplated. In an embodiment, the thermistor may be positioned behind the linear ultrasound transducer array forming part of the probe and coupled to an isolation box. The isolation box is configured to disable transmission of ultrasound signals from the ultrasound equipment by disabling the transmit circuitry by signaling the ultrasound equipment through a trigger mechanism such as a hardware interrupt. In particular, the isolation box may include a temperature sensing circuit for sensing a temperature of transducer array via the thermistor, and an imaging enable/freeze control circuit for disabling the transmit circuitry based on the temperature sensed by temperature sensing circuit. Other mechanisms could include disabling an array of multiplexers or transmit channel amplifiers commonly used in such circuits. Further disclosure of this embodiment is provided in U.S. patent application Ser. No. ______ (Attorney Docket No. 40036-0007) entitled Safety Systems And Methods For Ensuring Safe Use Of Intra-Cardiac Ultrasound Catheters which is filed concurrent with this application and is hereby incorporated by reference in its entirety.

Separate Cannula or Chest Tube

In an embodiment, an elongate support member in the form of a cannula or chest tube is placed into the thoracic cavity of a patient after making an incision. The cannula or chest tube may be of any size larger than the catheter and having a seal. Desirably, the cannula or chest tube is adapted to form a seal when the catheter is inserted, so as to avoid influx or efflux of gas, liquid or solid into the chest cavity of drainage of blood or serous fluids. A seal or valve (not shown) may be used for this purpose, as will be appreciated by a skilled artisan.

In another embodiment, a supporting portion of a catheter-receiving chest seal includes a separable part and a cutting device (e.g., trocar) by which the separable part can be removed. Once removed from the support member, the catheter receiving portion is located in a desired position, leaving a support member of reduced size attached to the catheter-receiving tube. After insertion of the support member, a catheter can be positioned in a desired location within a patient's body by inserting the catheter into the patient's body through the catheter-receiving tube at any time afterwards. A skilled artisan will appreciate that a variety of seals may be used to maintain the fluid integrity of the body space.

In another embodiment a separate cannula or chest tube is used to first form a hole leading into the chest cavity. A variety of cannula or chest tube designs may be used. For example, a large bore needle may be used to make an initial insertion, followed by a guide wire, removal of the needle and then an incision followed by a pleural access catheter and then cannula or chest tube.

Integrated Cannula or Chest Tube Catheter

In an embodiment a cannula or chest tube is integrated with a catheter. Upon insertion of the cannula portion, a catheter portion slides into the body and can be manipulated by a physician from outside the body. In an embodiment the catheter portion is removable from the cannula or chest tube portion. In another embodiment the cannula or chest tube portion includes a cutting edge or trocar device that is used to cut into the body for entry.

Disposable

Desirably the entire device (catheter or integrated cannula or chest tube catheter) is removed from a sterile package, connected to external equipment at a junction or connector and then discarded after one use. An integrated or separate disposable trocar may be used to breach an outside barrier to the thorax and establish access to the pericardium. All of these components may be packaged in a single sterile package. All of these components can be designed and packaged as a single use, disposable device.

Methods of Use

In a desirable embodiment a cannula or chest tube is inserted into a chest wall to access an interperitoneal space. The elongated body of a catheter having an ultrasound imaging sensor near the distal end of less than 50 cm, 45, 40, 35, 30, 25, 20 cm is inserted into the chest cavity through the cannula or chest tube and is manipulated with a handle of the catheter to bring a surface of the ultrasound imaging sensor near or in contact with the outer surface of the heart. Electric cables extending from the proximal end of the catheter are connected to an ultrasound driver/monitor equipment by means of a junction or connector. The ultrasound driver/monitor equipment receives the ultrasonic image information, stores the information and displays images as needed. Ultrasonic imaging then is carried out, preferably by the acquisition of a series of planer images from an ultrasonic phased array. The imaging portion of the catheter may be positioned on or near the exterior of the heart, over any chamber, by moving the catheter within the pericardium. Coupling of ultrasound energy between the transducer array and heart tissue occurs via pericardium serous fluid. In this manner, the ultrasound imaging catheter may be positioned a short distance from the surface of the heart so that the heart wall is beyond the region of near-zone interference commonly observed immediately adjacent to an ultrasound transducer surface.

Systems, Kits

In another embodiment, a cannula or chest tube is combined with a catheter in a single sterile unit system that inserts into an incision such that the catheter slides into a body space after insertion. The cannula or chest tube according to an embodiment has a seal. In another embodiment the cannula or chest tube has a flexible bag, balloon or other wrapper that forms a sterile boundary around the catheter as the catheter is pushed into the cannula or chest tube. This embodiment of the percutaneous catheter allows the use of a non-sterile catheter. This embodiment of the catheter generally does not use ECG electrode recording and may have an ultrasound transmitting fluid contacting the inner wall of the wrapper and the catheter surface, to allow ultrasonic energy transmission to and from the ultrasonic transducer array on the catheter.

In another embodiment a catheter or a system as described herein is packaged within a sterile wrapper or other sterile container for one time use. Desirably, a sterile wrapper is employed that is removed by tearing. In another embodiment, a sterile bag having a sealed aperture envelopes the catheter. During use, the sealed aperture is placed over an opening to a cannula or chest tube and the catheter is then pushed through the cannula or chest tube. After insertion, the bag continues to surround a proximal handle portion of the catheter and allows manipulation of the catheter without compromising sterility.

Another embodiment is a kit comprising a cannula or chest tube either separate or attached to a catheter, and a catheter in a container such as a box, plastic container or paper package. Optionally the catheter is packaged in a sterile wrapper such as a foil pack or plastic pack. The kit further may comprise a placard or paper instruction sheet.

Another embodiment comprises an imaging ultrasound percutaneous catheter according to various embodiments combined with thoracoscopic equipment, preferably with robotic thoracoscopic equipment that permits remote manipulation of the imaging portion of the catheter within the pericardium. Combined in such a system may be optical imaging capability and remote surgical equipment that permits microsurgery to be conducted while the heart and surgical implements are imaged and monitored by the ultrasound imaging catheter. The combination of embodiments of the present invention with thoracoscopic surgery is expected to provide better imaging of left ventricle myocardial segments to enable more accurate placement of leads for CRT and other procedures. In an embodiment, an electrode lead is placed in the tissue of the last contracting myocardial segment by microsurgery through the exterior of the left ventricle wall guided by ultrasound imaging, which may include tissue Doppler imaging, of the heart via an ultrasound imaging catheter within the pericardium and positioned on or near the heart.

Other combinations of the inventive features described above, of course easily can be determined by a skilled artisan after having read this specification, and are included in the spirit and scope of the claimed invention. References cited above are specifically incorporated in their entireties by reference and represent art known to the skilled artisan.

1. A peritoneal imager, comprising:
an elongated body having a distal end and a length less than about 20 inches and adapted for insertion through a cannula into a peritoneal space; and
an ultrasound transducer array coupled to the elongated body near the distal end suitable for ultrasound echocardiography.

2. An imager as described in claim 1, wherein the imaging array comprises multiple piezoelectric transducers, each connected by a coaxial cable to a proximal end of the elongated body.

3. An imager as described in claim 1, wherein the elongated body has a length of less than about 10 inches.

4. An imager as described in claim 1, wherein the ultrasonic transducer array comprises one of 48, 64, 96, or 128 transducers.

5. An imager as described in claim 1, wherein the elongated body is rigid and can be manipulated within a patient by moving a portion of the elongated body extending outside of the cannula.

6. An imager as described in claim 1, wherein a portion of the elongated body is bendable with a bend being controllable from a handle coupled to a proximal end of the elongated body.

7. An imager as described in claim 1, wherein the elongated body is configured to be manipulated by a robotic system.

8. An imager as described in claim 1, further comprising one or more electrodes.

9. An imager as described in claim 3, further comprising a coupling circuit configured to electrically isolate direct current between the piezoelectric devices in the elongated body and equipment connected to the imager.

10. An imager described in one of claim 1, 2, and 4, wherein the ultrasonic transducer array is a linear phased array transducer.

11. An integrated cannula and imaging catheter, comprising:
a sheath and an elongated body within the sheath slideably adapted for insertion through a chest wall into a peritoneal space;
a distal tip on the elongated body; and
an ultrasonic imaging array positioned on the elongated body proximal to the distal tip configured for obtaining a two dimensional image.

12. An integrated cannula and imaging catheter as described in claim 11, wherein the sheath comprises an extracorporeal fixation device, located external to the patient to prevent inward movement of the sheath.

13. An integrated cannula and imaging catheter as described in claim 11, wherein the sheath comprises an internal valve or seal.

14. An integrated cannula imager as described in claim 11, wherein the elongated body is less than 30 cm long.

15. An integrated cannula imager as described in claim 11, further comprising one or more ECG electrodes on the elongated body.

16. The imager as described in claim 1, wherein the imager is a single use, disposable device.

17. A method of using the imager of claim 1, comprising introducing a cannula into the wall of a patient's chest, inserting the elongated body into the cannula, and moving the inserted elongated body to a position near the heart.

18. The method of claim 17, wherein the position near the heart is within the pericardium.

19. A method of imaging a heart of a patient using an ultrasound imaging sensor positioned near a distal end of a catheter, comprising:
inserting a cannula into a thorax of the patient, the cannula having a seal;
inserting the ultrasound imaging sensor through the cannula into a peritoneal space within the patient;
positioning the ultrasound imaging sensor near the heart of the patient; and
collecting ultrasound image information by emanating ultrasound from the ultrasound imaging sensor and receiving ultrasound echoes with the ultrasound imaging sensor.

20. The method of claim 17, wherein the method is used to obtain heart images for use in a medical procedure selected from the group comprising a procedure to ablate heart tissue, a procedure to place permanent or temporary pacing or defibrillation leads, a procedure to repair or replace a heart valve, a procedure to modify the left atrial appendage, a procedure to modify or repair the atrial septal wall, a procedure to place or inject medicines or animal cells, a procedure to apply reperfusion therapy with laser or other tools, a procedure to remove or isolate heart tumors or infarcted tissue, a procedure to remove permanently implanted pacemaker leads, a procedure to measure cardiac output, a procedure to measure heart valve leakage and a procedure to diagnose and treat diseases or malfunctions of the heart.
